(19) United States
(12) Patent Application Publication
(10) Pub. No.: US 2011/0129437 A1
(43) Pub. Date: Jun. 2, 2011

(54) PLATELET ADHESION-RESISTANT MATERIAL

(75) Inventors: Ying-nan TSAI, Taipei (TW); Ken-Yuan Chang, Taipei (TW); Fa Chen Chi, Taipei (TW); Po-Yang Chen, Taipei (TW); Cheng-Tar Wu, Taipei (TW)

(73) Assignee: FAR EASTERN NEW CENTURY CORPORATION.

(21) Appl. No.: 12/956,179

(22) Filed: Nov. 30, 2010

(30) Foreign Application Priority Data
Dec. 1, 2009 (TW) 098141008

(51) Int. Cl. A61K 31/785; A61P 7/02; C08F 222/40; C08G 71/04

(52) U.S. Cl. 424/78.37; 526/306; 528/322

(57) ABSTRACT

A platelet adhesion-resistant material is provided, which includes polytriuret-urethane consisting essentially of repeating structural units of formulae (I) to (III) in a random order, in which when the total number of the three repeating structural units in the polytriuret-urethane is 100, the number of the repeating structural units (I) is about 5 to about 50:

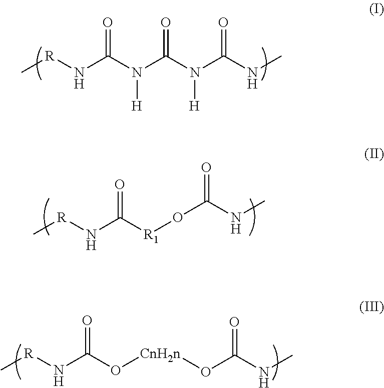

in which each R independently represents a C₂-C₁₆ alkylene group, a C₆-C₃₀ aromatic group, a C₆-C₃₀ alicyclic group; n is an integer of 2 to 16; and R₁ represents —(OCₘH₂ₘ)ₚ, in which m is an integer of 2 to 5, and p is an integer of 3 to 150.

### FIELD OF THE INVENTION

The present invention relates to a novel platelet adhesion-resistant polyurethane material which is applicable in the technical field of medical apparatus, especially in medical catheters as a material for the medical catheters per se or as a surface treatment resin for the medical catheters, to achieve platelet adhesion-resistant effect.

### DESCRIPTION OF THE PRIOR ART

The blood in human body is neither coagulated nor obstructed under normal conditions. However, when a foreign body, for example, medical polymer material, invades the human body, the flowing state of the blood and the nature of the vessel wall will definitely change. Additionally, if a material (such as acidic or alkaline substance) is dissolved from the material and enters the blood, the nature of the blood will also change. Such factors are likely to cause the blood to develop thrombosis, resulting blood obstruction, which often occurs during treatment of patients and bring huge secrete worry for medical treatment. Currently, the commonly used medical material is polyurethane (PU), which has good biocompatibility compared with silicone and polyvinylchloride (PVC), but doe not have good platelet adhesion-resistant properties.

Presently, the method of improving the platelet adhesion-resistant properties of PU wider research mainly includes chemical and physical improvement to modify the properties of the material, so as to achieve the platelet adhesion-resistant function.

As for physical improvement, in 1970, Lyman found in research that use of a microdomain structure of polyurethane-urea (PUU) could reduce the platelet adhesion effect (see D. J. Lyman, K. Knutson, and B. McNeil, Trans Am Soc Artif Intern Organs, 21: 49-53. (1975)). U.S. Pat. No. 4,687,831 also discloses that PUU of a microdomain structure synthesized from 4,4′-diphenylmethane-diisocyanate (MDI), poly(tetramethylene oxide) (PTMO), and 4,4′-diaminobenzanilide shows low platelet adhesion, has good anti-thrombosis and mechanical properties as elastomer, and thus is suitable as a material of artificial organs, such as blood vessel, kidney, and heart. It further discloses that the best platelet adhesion-resistant effect is achieved when the domain structure is in the range of 10-20 nm. Although PU has high biocompatibility compared with other polymer materials, it still causes platelet adhesion and thus thrombosis.

Another method to achieve platelet adhesion-resistance is chemical modification, that is, the PU material is surface modified to introduce molecules having specific functions, such as natural anticoagulation substance, hydrophilic groups and/or anionic functional groups, so as to further improve the biocompatibility between the material and the blood. Such surface modification includes the following.

(1) Bio-Mimesis of Material Surface

The most common method is to introduce a natural anticoagulant factor heparin onto the surface of a polymer material. The main mechanism is that heparin can combine with antithrombin III in the blood to form a complex, thus inhibiting the initiation of the coagulation factor to achieve the anticoagulation effect (see J. Fareed, Seminars in Thrombosis and Hemostasis, 11(1): 1-9 (1985)). Furthermore, by introducing, for example, albumin (see M. Munro, A. J. Quattrone, S. R. Ellsworth, P. Kulkarni, American Society for Artificial Internal Organs, 27:499-503 (1981)) or diionic material, such as phosphorylcholine (PC) (see K. Ishihara, R. Aragaki, T. Ueda, A. Watenabe and N. Nakabayashi, J. Biomed. Mater. Res. 24, 1069 (1990)), the biocompatibility of the material can also be improved, thus achieving the platelet adhesion-resistant effect.

(2) Material Surface with Hydrophilicity

The most common method is to introduce a hydrophilic group, such as polyethylene glycol (PEG), polyethylene oxide (PEO) (see D. K. Han, S. Y. Jeong and Y. H. Kim, J. Biomed. Mater. Res. Appl. Biomater. 23(A2), 211. (1989); and K. D. Park, W. G. Kim, H. Hacobs, T. Okano and S. W. Kim, J. Biomed. Mater. Res. 26, 739 (1992)) onto the surface of a common material by plasma or chemical grafting method. This is based on the fact that PEG itself is not toxic and has good biocompatibility. By introducing hydrophilic PEG or PEO onto the surface of the material, fluffy swing is formed on the surface of the material, thus reducing platelet adhesion, and achieving the antithrombotic effect.

(3) Material Surface with Negative Charges

Because the platelet in the blood is negatively charged in nature, and because like charges repel each other, some researches suggest that if the electronegativity on the surface of a material is increased, the platelet adhesion-resistant effect can be achieved. It is also reported that in this method, if a functional group having negative ion, such as sulfonate anion, is introduced onto a terminal of the hydrophilic group PEG, the material will exhibit bioactivity similar to that of the natural anticoagulation substance heparin in the blood, and can also exhibit good platelet adhesion-resistant effect (see J. Jozefonvicz and M. Jozefowicz, J. Biomater. Sci. Polymer Edn 1, 147 (1990); D. K. Han, N. Y. Lee, K. D. Park, Y. H. Kim, H. I. Cho and B. G. Min, Biomaterials 16, 467 (1995); K. D. Park, W. K. LEE, J. E. LEE, Y. H. KIM, ASAIO Journal. 42(5): 876-880 (1996); and D. K. Han, K. D. Park, Y. H. Kim, J. of Biomaterials Science-Polymer Edition, 9(2): 163-174. (1998)). The present invention proposes a novel polytriuret-urethane (PTU) material mainly on the basis of the argument that a material having negative charges on its surface will have platelet adhesion-resistant effect. As the PTU material contains the triuret repeating structural units of special formula (I), it can improve the electronegativity of the material, and since like charges repel each other, the platelet will not be easily adhered to the surface of the PTU material, thus achieving good platelet adhesion-resistant effect without additional grafting and modification.

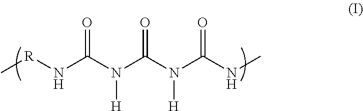

### SUMMARY OF THE INVENTION

Accordingly, the present invention is directed to a platelet adhesion-resistant material which includes polytriuret-urethane consisting essentially of repeating structural units of formulae (I) to (III) in a random order, in which when the total number of the three repeating structural units in the polytriuret-urethane is 100, the number of the repeating structural units (I) is about 5 to 50:

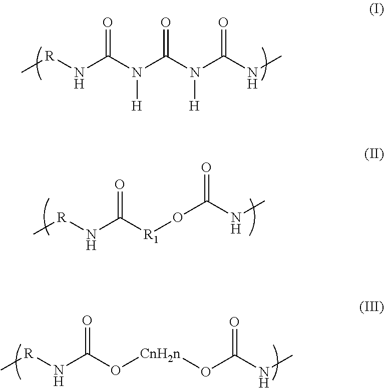

in which each R independently represents a C₂-C₁₆ alkylene group, a C₆-C₃₀ aromatic group, or a C₆-C₃₀ alicyclic group; n is an integer of 2 to 16; and R₁ represents —(OCₘH₂ₘ)ₚ, in which m is an integer of 2 to 5, and p is an integer of 3 to 150.

The present invention is further directed to a platelet adhesion-resistant material which includes a polytriuret-urethane synthesized from urea; a diisocyanate selected from the group consisting of C₂-C₁₆ aliphatic diisocyanate, C₆-C₃₀ aromatic diisocyanate, C₆-C₃₀ alicyclic diisocyanate, and a combination thereof; a C₂-C₁₆ glycol; and a polyglycol, in which the equivalent ratio of the urea to the glycol and the polyglycol is about 1:1 to about 1:19.

The platelet adhesion-resistant material of the present invention can be used in medical catheters as a material for the medical catheters per se or as a surface treatment resin for the medical catheters.

### BRIEF DESCRIPTION OF THE DRAWINGS

FIG. 1 shows the results of the platelet adhesion experiment.

### DETAILED DESCRIPTION

The present invention provides a polytriuret-urethane (PTU) material mainly on the basis of the argument that a material having negative charges on its surface will have platelet adhesion-resistant effect. As the PTU material itself has the triuret repeating structural units of special formula (I) and can thus improve the electronegativity, and as like charges repel each other, the platelets will not be easily adhered to the surface of the polytriuret-urethane material of the present invention, thus achieving good platelet adhesion-resistant effect.

It is well known that the main structure of a common polyurethane material is synthesized from diisocyanate, polyglycol, and glycol. The main feature of the present invention is to replace part of the glycol and polyglycol with urea for synthesis of polyurethane, so that the resulting PTU has triuret chains with negative charges, so as to achieve the platelet adhesion-resistant effect. The PTU material of the present invention has high biocompatibility, thus improving the industrial applicability and safety of medical equipment. Furthermore, no additional grafting or modification is needed for the material, thus reducing the manufacturing cost and improving the convenience of application.

The PTU of the present invention substantially consists of the repeating structural units of formulae (I), (II), and (III) in a random order, in which when the total number of the three repeating structural units in the polytriuret-urethane is 100, the number of the repeating structural units (I) is about 5 to 50:

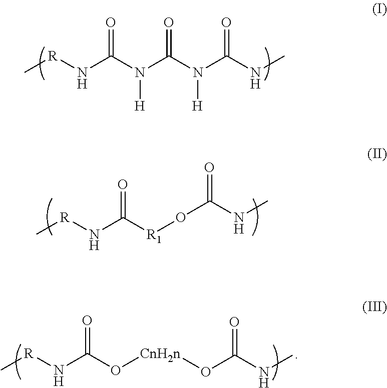

The PTU of the present invention contains the triuret repeating structural units of special formula (I), which allows it to generate negative charges. As the N atom in the structure (I) is connected to two electron-withdrawing groups, N—H bond is likely to be deprotonated in neutral or weak alkaline environment to generate a compound of structure (IV), so that the PTU material of the present invention has negative charges and the electronegativity of the material itself is improved. Since like charges repel each other, the platelet will not be easily adhered to the surface of the polytriuret-urethane material of the present invention.

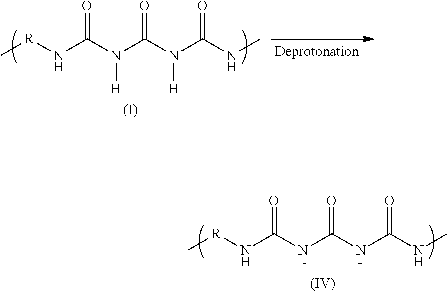

In chemical formulae (I) to (IV) above, each R independently represents a C₂-C₁₆ alkylene group, a C₆-C₃₀ aromatic group, or a C₆-C₃₀ alicyclic group; n is an integer of 2 to 16, preferably an integer of 2 to 10, and most preferably an integer of 3 to 6; and R₁ represents —(OCₘH₂ₘ)ₚ, in which m is an integer of 2 to 5, and p is an integer of 3 to 150, preferably an integer of 3-100, and more preferably an integer of 10-50.

According to the present invention, the term “C₂-C₁₆ alkylene group” refers to a C₂-C₁₆ straight chain or branched chain saturated divalent hydrocarbon moiety, preferably C₂-C₁₂ straight chain or branched chain saturated divalent hydrocarbon moiety, and more preferably C₂-C₆ straight chain or branched chain saturated divalent hydrocarbon moiety. Exemplary alkylene groups include, but are not limited to, hexamethylene, 1,6-hexylene, butylene, trimethylhexamethylene, and the like.

According to the present invention, the term “C₆-C₃₀ aromatic group” refers to a C₆-C₃₀ divalent unsaturated hydrocarbon moiety with an unsaturated aromatic ring, and preferably C₆-C₁₅ divalent unsaturated hydrocarbon moiety with an unsaturated aromatic ring. Exemplary aromatic groups include, but are not limited to, phenylene, 4,4′-methylenediphenyl, tolylene, naphthylene, and the like.

According to the present invention, the term “C₆-C₃₀ alicyclic group” refers to a C₆-C₃₀ divalent saturated hydrocarbon moiety with a saturated carbon ring, and preferably C₆-C₁₅ divalent saturated hydrocarbon moiety with a saturated carbon ring. Exemplary alicyclic groups include, but are not limited to, cyclohexylene, 4,4′-methylenedicyclohexyl,

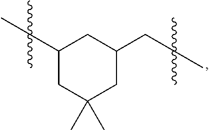

and the like.

The platelet adhesion-resistant PTU material of the present invention is a polyurethane material synthesized from urea, polyglycol, glycol, and diisocyanate, with a molecular weight of 10,000-200,000, preferably 30,000-150,000, and most preferably 40,000-100,000. As well known to those of ordinary skill in the art, the main structure of a common polyurethane material is synthesized from a diisocyanate, a polyglycol, and a glycol. The main feature of the present invention is that part of glycol and polyglycol is replaced by urea to synthesize PTU according to the conventional polyurethane synthesis process, so that the resulting PTU has the triuret repeating structural units of formula (I) with negative charges, thus achieving the platelet adhesion-resistant effect. In the preparation, the equivalent ratio of urea to glycol and polyglycol is about 1:1 to about 1:19, and preferably about 1:1.8 to 1:6.

According to the present invention, the glycol is a C₂-C₁₆ glycol, and preferably C₂-C₁₀ glycol. Exemplary glycols include, but are not limited to, ethylene glycol, propylene glycol, butylene glycol, pentanediol, hexanediol, and a derivative or combination thereof.

Useful polyglycols in the present invention include, but are not limited to, polyethylene glycol, poly(propylene glycol) (PPG), poly(tetramethylene glycol) (PTMEG), and a derivative or combination thereof. According to an embodiment of the present invention, the used polyglycol has a molecular weight of 200-9,000, preferably 200-5,000, and more preferably 200-2,000.

According to the present invention, useful diisocyanates include C₂-C₁₆ aliphatic diisocyanate, C₆-C₃₀ aromatic diisocyanate, C₆-C₃₀ alicyclic diisocyanate, and a derivative and combination thereof. Preferred aliphatic diisocyanates include, but are not limited to, hexamethylene diisocyanate (HDI), 1,6-hexylene diisocyanate, tetramethylene diisocyanate, tri ethylhexamethylene diisocyanate, or a derivative thereof. Preferred aromatic diisocyanates include, but are not limited to, diphenylmethane-4,4′-diisocyanate (MDI), toluene diisocyanate (TDI), 1,5-naphthalene diisocyanate (NDI), p-phenylene diisocyanate (PPDI), or a derivative thereof. Preferred alicyclic diisocyanates include, but are not limited to, cyclohexane diisocyanate, isophorone diisocyanate (IPDI), dicyclohexylmethane diisocyanate (H₁₂MDI), or a derivative thereof.

The following embodiments are intended to further illustrate the present invention, but are not to limit the scope of the present invention. All modifications and changes which can be easily made by one of ordinary skill in the art are within the scope of the disclosure of this specification and the appended claims.

### Embodiments

### Chemicals for Synthesis

Diphenylmethane-4,4′-diisocyanate (MDI, 98%), dicyclohexylmethane diisocyanate (H₁₂MDI, 90%), isophorone diisocyanate (IPDI, 98%), polyethylene glycol (PEG; Avg. Mn˜2000), 1,4-butanediol (BD, 99%), urea (99.0-100.5%), and polyethyleneimine (PEI), are commercially available from Sigma; Eastman 58245 is commercially available from Noveon; dimethyl acetamide (DMAC, reagent grade) is commercially available from TEDIA, and is subjected to distillation to obtain fresh DMAC before the reaction.

### Synthesis of PTU

### Example 1

### Synthesis of PTU Containing Urea 15% (PTU1)

1 equivalent polyethylene glycol was added into a 500 ml four-necked reaction flask, placed in a vacuum oven before reaction and heated to 100° C., and dehydrated at a vacuum degree of 1 torr for 8 h. 30 ml fresh DMAC was added, and further dehydrated at 60° C. and a vacuum degree of 1 torr for 2 h. 2.4 equivalent 1,4-butanediol was added, and the temperature was raised to 80° C., and the temperature equilibrium was reached about half an hour later. Then, 0.6 equivalent urea was added, followed by 4 equivalent MDI for polymerization. MDI had to be added in portions, and each portion was 0.02-0.05 equivalents. Furthermore, when MDI was added, the viscosity would increase, so it was necessary to add DMAC for dilution to prevent the generation of gel. The cycling step of adding MDI, and diluting when the viscosity of the polymer increased was repeated until the viscosity of the polymer no longer increased, then methanol was added to quench the reaction, and the product was precipitated in ice water.

### Example 2

### Synthesis of PTU Containing Urea 25% (PTU2)

1 equivalent polyethylene glycol was added into a 500 ml four-necked reaction flask, placed in a vacuum oven before reaction and heated to 100° C., and dehydrated at a vacuum degree of 1 torr for 8 h. 30 ml fresh DMAC were added, and further dehydrated at 60° C. and a vacuum degree of 1 torr for 2 h. 2 equivalent 1,4-butanediol was added, and the temperature was raised to 80° C., and the temperature equilibrium was reached about half an hour later. Then, 1 equivalent urea was added, followed by 4 equivalent MDI for polymerization. MDI had to be added in portions, and each portion was 0.02-0.05 equivalent. Furthermore, when MDI was added, the viscosity would increase, so it was necessary to add DMAC for dilution to prevent the generation of gel. The cycling step of adding MDI, and diluting when the viscosity of the polymer increased was repeated until the viscosity of the polymer no longer increased, then methanol was added to quench the reaction, and the product was precipitated in ice water.

### Example 3

### Synthesis of PTU Containing Urea 35% (PTU3)

1 equivalent polyethylene glycol was added into a 500 ml four-necked reaction flask, placed in a vacuum oven before reaction and heated to 100° C., and dehydrated at a vacuum degree of 1 torr for 8 h. 30 ml fresh DMAC were added, and further dehydrated at 60° C. and a vacuum degree of 1 torr for 2 h. 1.25 equivalent 1,4-butanediol was added, and the temperature was raised to 80° C., and the temperature equilibrium was reached about half an hour later. Then, 1.25 equivalent urea was added, followed by 3.5 equivalent MDI for polymerization. MDI had to be added in portions, and each portion was 0.02-0.05 equivalents. Furthermore, when MDI was added, the viscosity would increase, so it was necessary to add DMAC for dilution to prevent the generation of gel. The cycling step of adding MDI, and diluting when the viscosity of the polymer increased was repeated until the viscosity of the polymer no longer increased, then methanol was added to quench the reaction, and the product was precipitated in ice water.

### Example 4

### Synthesis of PTU Containing Urea 35% (PTU4)

1 equivalent polyethylene glycol was added into a 500 ml four-necked reaction flask, placed in a vacuum oven before reaction and heated to 100° C., dehydrated at a vacuum degree of 1 torr for 8 h. 30 ml fresh DMAC were added, and further dehydrated at 60° C. and a vacuum degree of 1 torr for 2 h. 1.25 equivalent 1,4-butanediol was added, and the temperature was raised to 80° C., and the temperature equilibrium was reached about half an hour later. Then, 1.25 equivalent urea was added, followed by 3.5 equivalent H₁₂MDI for polymerization. MDI had to be added in portions, and each portion was 0.02-0.05 equivalent. Furthermore, when MDI was added, the viscosity would increase, so it was necessary to add DMAC for dilution to prevent the generation of gel. The cycling step of adding MDI, and diluting when the viscosity of the polymer is raised was repeated until the viscosity of the polymer no longer increased, then methanol was added to quench the reaction, and the product was precipitated in ice water.

### Example 5

### Synthesis of PTU Containing Urea 35% (PTU5)

1 equivalent polyethylene glycol was added into a 500 ml four-necked reaction flask, placed in a vacuum oven before reaction and heated to 100° C., dehydrated at a vacuum degree of 1 torr for 8 h. 30 ml fresh DMAC were added, and further dehydrated at 60° C. and a vacuum degree of 1 torr for 2 h. 1.25 equivalent 1,4-butanediol was added, and the temperature was raised to 80° C., and the temperature equilibrium was reached about half an hour later. Then, 1.25 equivalent urea was added, followed by 3.5 equivalent IPDI for polymerization. MDI had to be added in portions, and each portion was 0.02-0.05 equivalent. Furthermore, when MDI was added, the viscosity would increase, so it was necessary to add DMAC for dilution to prevent the generation of gel. The cycling step of adding MDI, and diluting when the viscosity of the polymer increased was repeated until the viscosity of the polymer no longer increased, then methanol was added to quench the reaction, and the product was precipitated in ice water.

### Comparative Example 1

### PU1

Eastman 58245 sold by Noveon was dissolved in DMAC (about 20 wt %) by heating to 80° C. as sample of this example.

### Comparative Example 2

### PU2

PU was synthesized according to the PU synthesis technology disclosed in U.S. Pat. No. 4,687,831 as sample of this example.

### Comparative Example 3

### PEI

PEI sold by Sigma was used as sample of this example.

Film-Forming Method of Samples

Film-Forming of PTU

Polytriuret-urethanes synthesized from Examples 1 to 5 were dissolved in DMAC (about 20 wt %) by heating. Next, polymer-containing DMAC was coated into a film, and placed in an oven of 90° C. for 2 h to remove the solvent DMAC, giving dry PTU film.

Film-Forming of PU

The polyurethane materials of Comparative Examples 1 to 2 were dissolved in DMAC (about 20 wt %) by heating. Next, polymer-containing DMAC was coated into a film, and placed in an oven of 90° C. for 2 h to remove the solvent DMAC, giving dry PU film.

Film-Forming of PEI

The sample of Comparative Example 3 was coated into a film, and placed in an oven of 90° C. for 2 h, giving dry PEI film.

Determination of Surface Potential

The PTU film was frozen to dryness and pulverized into powder. Then the surface potential of the powder was measured, so as to verify the surface electrical properties of PTU and observe the variation of the surface electrical properties with the urea content.

Experimental Results

It can be seen from Table I that, compared with common, commercially available polyurethane materials, the polytriuret-urethane (PTU) synthesized from urea according to the present invention definitely has high electronegativity. Furthermore, the higher the urea content, the higher the electronegativity of the PTU, and the more the negative charges carried. The negative charges will repel the negative charges in the platelet, so that the platelet adhesion rate is reduced, thereby achieving the platelet adhesion-resistant function.

TABLE 1

| | Urea content | Zeta Potential |
|---|---|---|
| | (%) | (mv) |
| PU | 0 | −17.49 |
| Example 1 (PTU1) | 15 | −24.23 |
| Example 2 (PTU2) | 25 | −24.34 |
| Example 3 (PTU3) | 35 | −25.61 |
| Example 4 (PTU4) | 35 | −24.93 |
| Example 5 (PTU5) | 35 | −25.02 |

### Platelet Adhesion Experiment

### Examples 1 to 5

### Step 1

Fresh porcine plasma was separated with centrifuge (1500 rpm; 15 min), to get plasma poor platelet (PPP) with a platelet content of 17×10³-20×10³ per μl.

### Step 2

The filmed PTU material was cut into pieces of 1 cm² and washed with PBS buffer, and then the PTU piece was fixed on a glass plate.

### Step 3

Fresh PPP 1 ml was covered on the surface of the PTU, and after standing at room temperature for 2 h, the PPP was aspirated. The number of the platelet remaining in the PPP was calculated with a blood cell counter, and the adsorption of the platelet by the material was calculated by the equation below.

$$Platelet adsorption rate ( \% ) = \frac{Number of unadsorbed platelet - number of adsorbed platelet}{number of unadsorbed platelet} \times 100$$

### Comparative Examples 1 to 3

The platelet adhesion of the PU film and the PEI film was tested in the same manner as Steps 2 and 3 of Examples 1 to 5, as control groups.

Results of Platelet Adhesion Experiment

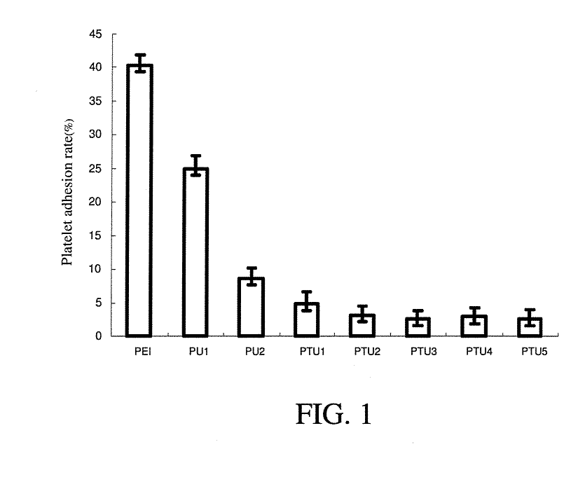

In the platelet adhesion experiment, PEI material with positively charged surface, to which platelet is likely to adhere, and commonly used PU material were used as control groups for the material of the present invention, and the experimental results are as shown in FIG. 1. It can be seen in FIG. 1 that, as it has more negative charges on the surface, PTU has better platelet adhesion-resistant effect than common PU and PEI having positive charges on the surface. It can also be seen that the platelet adhesion-resistant effect of PTU will increase with the increase of the urea content, and the electronegativity of PTU is high. Higher electronegativity means more negative charges, which will repel the negative charges in the platelet, so that the platelet adhesion rate is reduced, thereby achieving good platelet adhesion-resistant effect. Thus, the lower the platelet adsorption rate, the better the platelet adhesion-resistant effect.

It can be easily understood that various modifications of the present invention are feasible and can be easily envisioned and expected by those skilled in the art.

1. A platelet adhesion-resistant material, comprising polytriuret-urethane consisting essentially of repeating structural units of formulae (I) to (III) in a random order, wherein when the total number of the three repeating structural units in the polytriuret-urethane is 100, the number of the repeating structural units (I) is about 5 to 50: 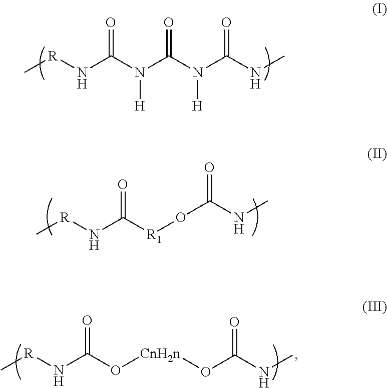
wherein each R independently represents a C₂-C₁₆ alkylene group, a C₆-C₃₀ aromatic group, or a C₆-C₃₀ alicyclic group; n is an integer of 2 to 16; and R₁ represents —(OCₘH₂ₘ)ₚ, wherein m is an integer of 2 to 5, and p is an integer of 3 to 150.

2. The platelet adhesion-resistant material according to claim 1, wherein each R independently represents a C₂-C₁₂ alkylene group, a C₆-C₁₅ aromatic group, or a C₆-C₁₅ alicyclic group; n is an integer of 2-10; and p is an integer of 3-100.

3. The platelet adhesion-resistant material according to claim 1, wherein each R independently represents a C₂-C₆ alkylene group, a C₆-C₁₅ aromatic group, or a C₆-C₁₅ alicyclic group; n is an integer of 3-6; and p is an integer of 10-50.

4. The platelet adhesion-resistant material according to claim 1, wherein each R independently represents hexamethylene, 1,6-hexylene, butylene, trimethylhexamethylene, phenylene, 4,4′-methylenediphenyl, tolylene, naphthylene, cyclohexylene, 4,4-methylenedicyclohexyl, or

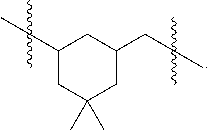

5. The platelet adhesion-resistant material according to claim 1, wherein the polytriuret-urethane has a molecular weight of 10,000-200,000.

6. The platelet adhesion-resistant material according to claim 5, wherein the molecular weight of the polytriuret-urethane is 30,000-150,000.

7. The platelet adhesion-resistant material according to claim 6, wherein the molecular weight of the polytriuret-urethane is 40,000-100,000.

8. A method of treating a medical catheter per se or as a surface treatment resin for medical catheters which comprises applying thereto or incorporating therein the platelet adhesion-resistant material according to claim 1.

9. A platelet adhesion-resistant material, comprising a polytriuret-urethane synthesized from urea; a diisocyanate selected from C₂-C₁₆ aliphatic diisocyanate, C₆-C₃₀ aromatic diisocyanate, C₆-C₃₀ alicyclic diisocyanate, and a combination thereof; a C₂-C₁₆ glycol; and a polyglycol, wherein the equivalent ratio of the urea to the glycol and the polyglycol is about 1:1 to about 1:19.

10. The platelet adhesion-resistant material according to claim 9, wherein the C₂-C₁₆ aliphatic diisocyanate is hexamethylene diisocyanate (HDI), 1,6-hexylene diisocyanate, tetramethylene diisocyanate, trimethylhexamethylene diisocyanate, or a derivative thereof.

11. The platelet adhesion-resistant material according to claim 9, wherein the C₆-C₃₀ aromatic diisocyanate is diphenylmethane-4,4′-diisocyanate (MDI), toluene diisocyanate (TDI), 1,5-naphthalene diisocyanate (NDI), p-phenylene diisocyanate (PPDI), or a derivative thereof.

12. The platelet adhesion-resistant material according to claim 9, wherein the C₆-C₃₀ alicyclic diisocyanate is cyclohexane diisocyanate, isophorone diisocyanate (IPDI), dicyclohexylmethane diisocyanate (H₁₂MDI), or a derivative thereof.

13. The platelet adhesion-resistant material according to claim 9, wherein the C₂-C₁₆ glycol is ethylene glycol, propylene glycol, butylene glycol, pentanediol, hexanediol, or a derivative or combination thereof.

14. The platelet adhesion-resistant material according to claim 9, wherein the polyglycol is polyethylene glycol, poly(propylene glycol) (PPG), poly(tetramethylene glycol) (PTMEG), or a derivative or combination thereof.

15. The platelet adhesion-resistant material according to claim 14, wherein the polyglycol has a molecular weight of 200-9,000.

16. The platelet adhesion-resistant material according to claim 15, wherein the molecular weight of the polyglycol is 200-5,000.

17. The platelet adhesion-resistant material according to claim 16, wherein the molecular weight of the polyglycol is 200-2,000.

18. The platelet adhesion-resistant material according to claim 9, wherein the polytriuret-urethane has a molecular weight of 40,000-100,000.

19. The platelet adhesion-resistant material according to claim 9, wherein the equivalent ratio of the urea to the glycol and the polyglycol is about 1:1.8 to about 1:6.

20. A method of treating a medical catheter per se or as a surface treatment resin for medical catheters which comprises applying thereto or incorporating therein the platelet adhesion-resistant material according to claim 9.
